(19) United States
(12) Patent Application Publication
(10) Pub. No.: US 2005/0267394 A1
(43) Pub. Date: Dec. 1, 2005

(54) A-EP Pronounced as: APE, for: A-animal E-eye P-patch

(76) Inventor: Rose T. Mavritte, Washington, DC (US)

Correspondence Address: Rose T. Mavritte, p o bx 29354, Washington, DC, 20017, US

(21) Appl. No.: 10/769,653

(22) Filed: Jun. 1, 2004

(51) Int. Cl. A61F 13/12

(52) U.S. Cl. 602/74

(57) ABSTRACT

Learning, after getting my chow chow's, they ( and other pets ) can and do have eye problems introduced me to the enormous world of veterinary opthomology and; need for an item like my invention of A-EP, pronounced APE, for, A-animal E-eye P-patch. Two of my dogs began having eye problems taking me into the vast world of veterinary opthomology. Seeing angry eye conditions I realized need for protection cover-care. My largest chow was without any visible eye problems, then suddenly he has need for two separate, different conditions eye surgeries. Initially I didn't know regular vets cannot do all, including attend pets eyes. As did the vet not know who to refer me. That ushered me into role of sleuth in the veterinary opthomology field. A world in and of itself. The field is so vast, when I did not relish a crass side of one, I had the luxury of looking further and found one even other veterinary opthomologists praise as; the best in the business. Taking two of my chow chows for eye care/attention in this specialty field, I saw many animal companions bought in needing care. Clientele having—one—angry/injured eye is tremendous in number. Each of all having -one good eye, can benefit with use of A-EP. There're many pets/animals coming to veterinary opthomologists offices that A-EP can provide protection. As can A-EP render relief for us, to not see our friends angry eye condition as we enjoy the otherwise happy side of their companionship. My initial consideration for A-EP, latter year 2000-early 2001. During one of my dogs visit to the vet, his eyes problematic I asked about them ( prior to surgery by the veterinary opthomologist) During his examining, to one of my specific questions the vet stopped, looking directly at us his answer; “Show me how to make eye protection for a dog.” That breathed life into my creating A-EP Finding nothing on the internet with my descriptive concepts prompted my invention of A-EP as protective cover-care. As did seeing the vast clientele in veterinary opthomologists offices, show need for protective care for many disturbing-angry-uncovered pet/animal eyes.

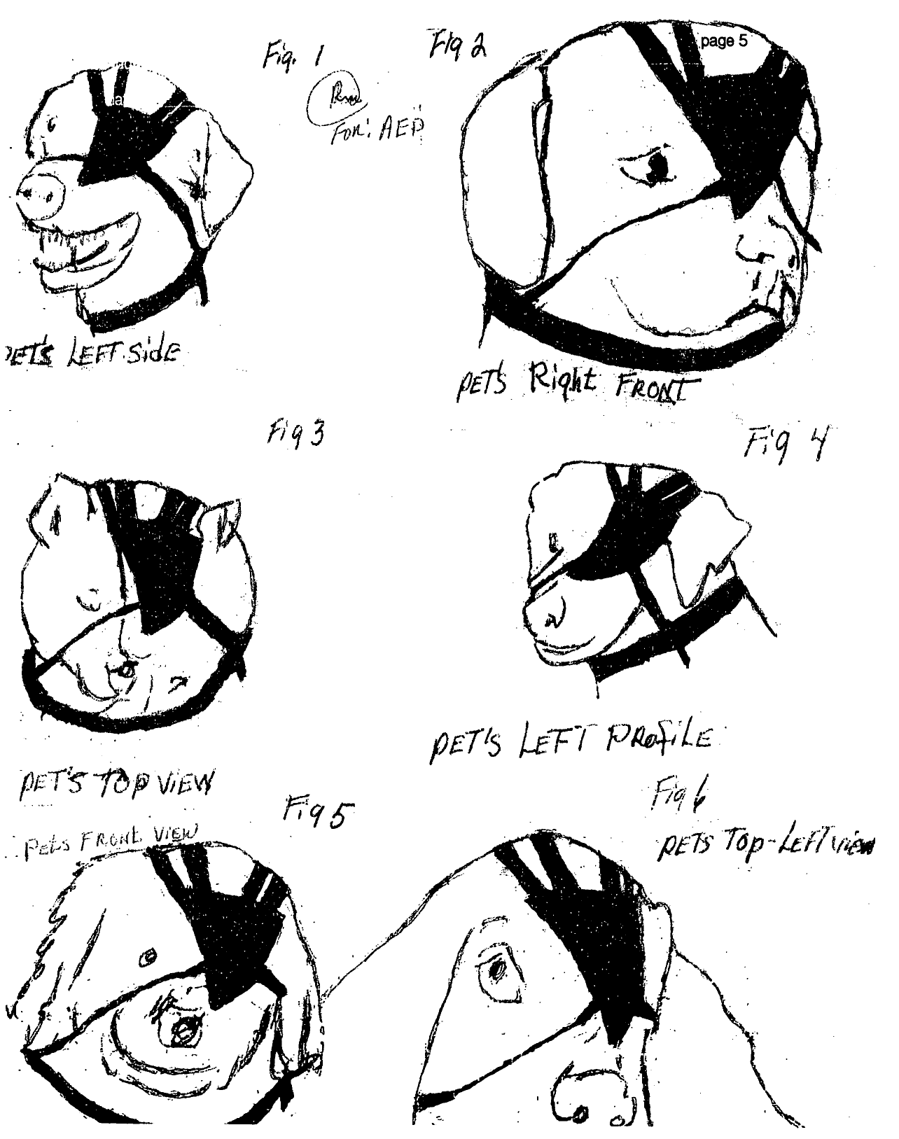

Diagram via: a. sketches-drawings b. pictures-photographs

Parts consist of I. Triangular eye patch II. Belt-straps III. Neck-Bard IV. Elasiticity

A-EP is meant for any and all pets/animals with a problematic eye.

A-EP'S Structure:
a. Denim double layered triangular eye patch-sized to fit individual pet
b. —6—belt straps strategically placed
c. One inch wide neck-band—or, wide as need for individual pet
d. One inch square heavy duty elastic (or, size to fit individual pet)

A-EP'S structured function

a A-EP'S triangular eye patch is denim double layered. Side to side 5¾ inches—or, wide as need for individual pet Top to triangular point 4½ inches—or, wide as need for individual pet.

b. Stitch one inch square elastic end to end with one inch wide neck-band (or; wide as need for pet size ) make it circular and—comfortably taut. Cover elastic with denim double layer.

c. —6—belt straps strategically placed. Each, ¼ inch within double layered triangular eye-patch. Stitch in place. Extend downward to neck-band. Stitch to neck-band, extend downward ⅛-¼ inch pas neck-band to grasp for adjustments

Like shoes A-EP must be—sized to fit. Band, belts and triangular patch must all be—sized for individual pet fit.

Each of —4—belt saps are at top-back of triangular eye-patch.
a FIRST of —4—belt-straps at; top-back of triangular eye-patch. Strategically place ¼-2. ½ inch from left corner extend 5″ ( Or, sized to fit ) from triangular eye patch to neck-band, stitch in place. Extend ¾ inch past neck-band: to grasp for adjusting. This belt-strap begins ¾ inch wide at top, ( or wide as need for individual pet ) gradually expands to 1 inch (or size to fit)
b. SECOND of —4—top back belt-straps 1⅛ inch wide ( or sized to meet pets individual physical need) Extend from triangular eye patch downward 4½ inches, (or size to fit individual pet) with space between first and second belt strap at top ¼ inch. 2 inches down space gradually expand to ¾ inch near neck-band ( for this size fit ) stitch to neck-band then continue downward (in measure to meet end, with first belt-strap ) to grasp for adjusting
c. THIRD top-back belt-strap 1 inch wide (or; sized to meet individual pet need) extend downward to neck-band, with space between second and third belt-strap at top 1½ inch, gradually decrease to 1 inch near neck-band for this size fit Stitch in place, then extend past neck-band ( in measure to meet end, with both first and second belt-strap ) to grasp for adjustments.
d. FOURTH top-back belt-strap has no space between it and 3rd belt-strap. 1 inch wide ( or size to fit individual pet) it extends 1¾ inch from triangular eye patch to neck-band, its outer side ends ¼ inch from right corner located—exact opposite corner from first belt-strap. Ad past neck-band ( in measure to meet end, with first, second, and third belt-strap) to grasp for adjustments
e. FIFTH belt-strap ½ inch wide ( or; wide as need for individual pet ) 2 inches from triangular eye-patch point ( or; measure for individual pet ) 1½ inch from 4th top-back belt-strap (or; measure for individual pet) extend past neck-band (in measure to meet end, even with all other belt-straps ) to grasp for adjustments.
f. SIXTH of —6—belt-straps ¼ inch from left side of triangular point-peak. This denim double layered belt-strap must be thin, strong, not cover, cross, nor interfere with pets mouth-function in any way. If breed has erect ears A-EP allows an ear between 2nd and 3rd belt-strap on pets—left. Other ear aside first belt-strap on pets right. Only—one—bet-strap on good eye side of triangle. This—FULL SIDE is open for good eye. Belt-strap on good eye side is 5½ inches long ( or, size to individual pet) after it's ¼ inch top is stitched within triangle patch, and after stitched to neck-band. Top of this 5½ inches ( exposed) belt-strap is; ¼-½ inch wide (or size to fit individual pet ) 1¼ inch down from triangle patch, this—thin—belt-strap width, decrease by—gradual taper, while making gradual and slight curve toward pets back. Then at 2½ inches before neck-band expand back to ¼-½ inch width, for remainder of ( exposed ) inches. Stitch to neck-band making it—comfortably taut Extend past neck-band (to meet even with all other belt-straps ) to grasp for adjustments.

Each piece—sized to fit individual pet (as shoes ) The more minuscule in width SIXTH belt-strap is: only belt-strap on good eye side.

Finished product A-EP, is for protective cover on any injured/damaged/infected pet (or otherwise animal) eye.

1. I invented A-EP heretofore pronounced APE for; A-animal E-eye P-patch, for the purpose of protecting any pet/animal who has encountered an eye condition/problem/injury to one eye.
